(19) United States
(12) Patent Application Publication
(10) Pub. No.: US 2022/0098651 A1
(43) Pub. Date: Mar. 31, 2022

(54) Method of Determining Secondary Structure of a Nucleic Acid

(71) Applicant: President and Fellows of Harvard College, Cambridge, MA (US)

(72) Inventors: Rees F. Garmann, Santa Cruz, CA (US); Timothy K. Chiang, Cambridge, MA (US); Vinothan N. Manoharan, Cambridge, MA (US); Ofer Kimchi, Cambridge, MA (US); Michael P. Brenner, Cambridge, MA (US)

(21) Appl. No.: 17/482,765

(22) Filed: Sep. 23, 2021

Related U.S. Application Data
(60) Provisional application No. 63/085,561, filed on Sep. 30, 2020.

(51) Int. Cl. C12Q 1/6837

(52) U.S. Cl. CPC C12Q 1/6837

(57) ABSTRACT

A method is disclosed for analyzing the secondary structure of a nucleic acid using a patch oligonucleotide and a probe oligonucleotide.

### FIELD OF THE INVENTION

The present invention relates to the field of nucleic acid analysis, in particular, analysis of the secondary structure of a nucleic acid.

### SEQUENCE LISTING

The instant application contains a Sequence Listing which has been submitted electronically in ASCII format and is hereby incorporated by reference in its entirety. Said ASCII copy, created on Oct. 14, 2021, with the file name of Sequence_Listing_010498_01440_ST25.txt and is 2.95 kilobytes in size.

### BACKGROUND OF THE INVENTION

Methods for determining RNA secondary structure using RNA-DNA binding are known. See Uhlenbeck, et al., Nature 225:5232, 1970; Kierzek, et al., Biochemistry 45:2, 2006; Liang, et al., Biochemistry 49:37, 2010. Chemical probing methods are also known. See Merino, et al., JACS 127:12, 2006 and Watts, et al. Nature 460:711, 2009). However, methods of analyzing secondary structure of a nucleic acid that are faster, that are more accurate, and that need not rely on RNA folding algorithms are needed.

### SUMMARY

The present disclosure provides a method of analyzing secondary structure of a nucleic acid, such as a ribonucleic acid (“RNA”) or a deoxyribonucleic acid (DNA). The term nucleic acid generally refers to a nucleic acid polymer as is known in the art. Nucleic acids of the present disclosure are generally understood to be a polymer of nucleotides or ribonucleotides, whether natural or synthetic. According to one aspect, a nucleic acid of the present disclosure includes secondary structure and the methods described herein are used to analyze secondary structure, for example, by identifying portions of the nucleic acid which may bind to each other through complementary nucleotides.

According to one exemplary aspect, the method uses a patch oligonucleotide (which may be referred to herein as a “patch” for ease of understanding) and a probe oligonucleotide (which may be referred to herein as a “probe” for ease of understanding) to determine whether the patch and probe corresponding binding sites are coupled or independent. The patch or probe may be DNA or RNA, for example. According to one aspect, the nucleic acid to be analyzed is combined with a patch oligonucleotide and placed under hybridization conditions and the patch oligonucleotide binds to, i.e. hybridizes with, a corresponding patch oligonucleotide binding site on the nucleic acid. The patch oligonucleotide is designed with respect to the primary structure of the nucleic acid, i.e. the nucleic acid sequence, to be fully or partially complementary to a corresponding portion of the nucleic acid. The term “patch” is used to illustrate that the binding site is covered or “patched” by the patch oligonucleotide hybridized thereto. The term “patched nucleic acid” refers to the nucleic acid with the patch oligonucleotide hybridized or bound thereto. The patched nucleic acid is then combined with a probe oligonucleotide and placed under hybridization conditions and the probe oligonucleotide binds to, i.e. hybridizes with, a corresponding probe oligonucleotide binding site on the patched nucleic acid. The term “probed nucleic acid” refers to the nucleic acid with the probe oligonucleotide hybridized or bound thereto. The probed nucleic acid may include a patch bound thereto or it may not include a patch bound thereto. The term “patched-probed nucleic acid” refers to a nucleic acid with both a patch and a probe bound thereto. The probe oligonucleotide is designed with respect to the primary structure of the nucleic acid, i.e. the nucleic acid sequence, to be fully or partially complementary to a corresponding portion of the nucleic acid. The term “probe” is used to illustrate whether the patch oligonucleotide binding site (“patch binding site”) on the nucleic acid is coupled with the probe oligonucleotide binding site (“probe binding site”) on the nucleic acid or whether the patch oligonucleotide binding site on the nucleic acid is independent of the probe oligonucleotide binding site on the nucleic acid. According to this aspect, the probe oligonucleotide “probes” the nature of the respective binding sites as being either coupled or independent thereby revealing information about the secondary structure of the nucleic acid. According to one exemplary aspect, whether the patch binding site and probe binding site are coupled or independent is determined by comparing the binding yield of the probe to the patched nucleic acid (“first binding yield”) with the binding yield of the probe to the nucleic acid without the patch (“second binding yield”). The nucleic acid without the patch may be referred to herein as a “naked nucleic acid”. The naked nucleic acid is the same nucleic acid but without the patch hybridized thereto. When the first binding yield is greater than, or significantly different from, the second binding yield, the patch binding site and the probe binding site are determined to be coupled and located on a common secondary structural element of the nucleic acid. When the first binding yield and the second binding yield are similar, i.e. within experimental error for example, the patch binding site and the probe binding site are determined to be independent and located on different secondary structural elements of the nucleic acid.

According to one aspect, the nucleic acid may be a long nucleic acid, i.e. of 200 nucleotides in length or greater, such as an RNA of longer than 200 nucleotides, and the methods described herein can determine coupling between a patch binding site and a probe binding site with a long nucleotide distance between them, i.e, between 50 to 1000 nucleotides, as an example.

According to one aspect, the method is carried out with a plurality of patch oligonucleotides and a plurality of probe oligonucleotides as described further herein to determine the difference in binding yields when a particular patch or set of patches is used and when a particular probe or set of probes is used. Accordingly, the disclosure contemplates determining a first binding yield for a set of many combinations of patch oligonucleotides and probe oligonucleotides, whether binding yields are compared for each member of a set of patch oligonucleotides and each member of a set of probe oligonucleotides, i.e. patched nucleic acid and probe oligonucleotide, or whether binding yields for a plurality of patch oligonucleotides and compared to a plurality of probe oligonucleotides, i.e. plurality-patched oligonucleotide and a probe oligonucleotide or a plurality of probe oligonucleotides. The disclosure is intended to encompass more than just use of a single patch and a single probe to determine secondary structure.

These and other embodiments will become apparent from the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates in schematic the use of a patch DNA oligonucleotide and a probe DNA oligonucleotide to identify coupling between an exemplary patch binding site and an exemplary probe binding site within secondary structure of a RNA.

FIG. 2 illustrates in schematic tiling of a plurality of 3′ to 5′ patch oligonucleotides along the 5′ to 3′ length of an RNA such as STMV RNA.

FIG. 3 is a binding spectrum which plots binding yield per probe DNA oligonucleotide.

FIG. 4 is a graph illustrating the difference between the binding spectrum obtained with a given patch DNA oligonucleotide and without a patch DNA oligonucleotide.

FIG. 5 illustrates a pattern produced by aligning each difference plot according to position of the patch DNA oligonucleotide binding site along the RNA primary sequence.

FIG. 6 is a two dimensional (2D) heat map of the patch DNA oligonucleotide binding site versus the probe DNA oligonucleotide binding site.

FIG. 7 is a graph of the adjacency matrix for the consensus secondary structure for STMV RNA.

FIG. 8 is an image of the consensus RNA secondary structure for STMV RNA.

FIG. 9 is an image of the full 1M array layout with the smaller rectangular subarrays.

FIG. 10 is an image of a subarray with boundaries shown as blue lines, and with dark and light visual aid features shown as black and red dots, respectively.

FIG. 11 is an image of the spot layout of the upper left corner of the 1M array, with visual aid features, probe features, and undefined (free) features shown.

FIG. 12 is an image of the text file defining the custom sequences (SEQ ID NOs.: 1-16) and spatial positions of the probes. The probes may be manufactured by an outside vendor, such as Agilent.

The figures should be understood to present illustrations of embodiments of the invention and/or principles involved.

### DETAILED DESCRIPTION OF EMBODIMENTS

Aspects of the present disclosure are directed to a method for determining secondary structure of a nucleic acid using a patch oligonucleotide and a probe oligonucleotide. The binding yield of the probe to a patched nucleic acid and the binding yield of the probe to a naked nucleic acid are compared. When the binding yield of the probe to a patched nucleic acid is increased relative to the binding yield of the probe to a naked nucleic acid, the patch is determined to facilitate binding of the probe which indicates that the patch binding site and the probe binding site are coupled. According to one aspect, binding by the patch to the patch binding site enhances binding of the probe to the probe binding site. According to one aspect, the patch binding site and the probe binding site comprise complementary base pairings.

According to one aspect, when the binding yield of the probe to a patched nucleic acid is similar to the binding yield of the probe to a naked nucleic acid, the patch binding site and the probe binding site are determined to be independent and located on different secondary structural elements of the nucleic acid.

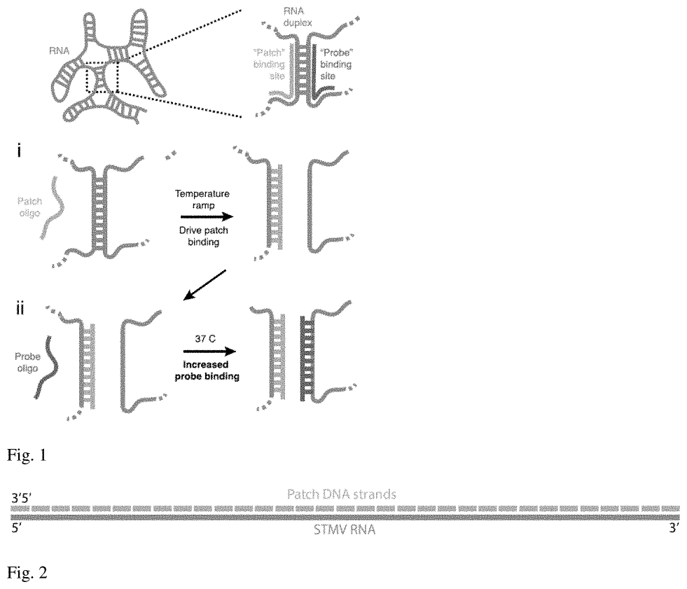

According to one aspect, the disclosure provides sequential binding of a short patch DNA strand to the nucleic acid followed by binding of a short probe DNA strand to the nucleic acid, which can affect the folded structure of the nucleic acid in a well-defined way. According to one exemplary aspect where the nucleic acid is a ribonucleic acid (“RNA”) and with reference to FIG. 1, (i) a short “patch” DNA strand is bound to the RNA by mixing the DNA and RNA and then heating and cooling the system. Then, (ii) a “probe” DNA strand is combined with the RNA bound with the patch DNA strand (“patched RNA”) and the yield of RNA-probe binding is measured. This binding yield is then compared to the binding yield of the probe DNA strand to that of naked RNA, i.e., RNA that has not been processed to include a patch DNA strand bound thereto. Increased binding of the DNA probe strand to the patched RNA indicates that the patch strand has “freed up” the binding site of the probe DNA strand, and, thus, that the patch and probe binding sites are coupled. Such coupling indicates that the binding sites reside on a common element of the RNA secondary structure. According to certain aspects, the methods described herein measure intramolecular base pair interactions directly. According to certain aspects, the intramolecular base pairings are analyzed to generate secondary structure of the nucleic acid. According to certain aspects, RNA folding algorithms known in the art may be used along with the methods described herein but are not required. Accordingly, methods described herein are carried out with the proviso that RNA folding algorithms are not used.

Aspects of the present disclosure contemplate that for a given patch and therefore a given patched nucleic acid, a plurality of probes are used to determine the binding of each individual one of the plurality of probes to the patched nucleic acid. The binding yield of each one of the plurality of probes to the given patched nucleic acid may be determined individually, i.e. a binding yield per individual probe. Similarly, the binding yield for each individual one of the plurality of probes may be determined with a naked nucleic acid.

Aspects of the present disclosure contemplate that for a given patch and therefore a given patched nucleic acid, a plurality of probes are used to determine the binding of the plurality of probes to the patched nucleic acid. The binding yield of the plurality of probes to the given patched nucleic acid may be determined, i.e. a binding yield for the plurality of probes to the patched nucleic acid. Similarly, the binding yield for the plurality of probes may be determined with a naked nucleic acid.

Aspects of the present disclosure contemplate that a plurality of patch nucleic acids may be used with a given nucleic acid to create a patched nucleic acid with a plurality of patches, referred to as a “plurality-patched nucleic acid.” The plurality-patched nucleic acid may then be combined with a probe and the binding yield determined. Similarly, the binding yield for the probe may be determined with a naked nucleic acid.

Aspects of the present disclosure contemplate that a plurality of patch nucleic acids may be used with a given nucleic acid to create a patched nucleic acid with a plurality of patches, referred to as a “plurality-patched nucleic acid.” The plurality-patched nucleic acid may then be combined with each individual one of a plurality of probes of each individual probe of the plurality of probes may be determined. Similarly, the binding yield for each individual one of the plurality of probes may be determined with a naked nucleic acid.

Aspects of the present disclosure contemplate that a plurality of patch nucleic acids may be used with a given nucleic acid to create a patched nucleic acid with a plurality of patches, referred to as a “plurality-patched nucleic acid.” The plurality patched nucleic acid may then be combined with a plurality of probes as described above. The binding yield of the plurality of probes to the given plurality-patched nucleic acid may be determined, i.e. a binding yield for the plurality of probes to the plurality-patched nucleic acid. Similarly, the binding yield for the plurality of probes may be determined with a naked nucleic acid.

One of skill will readily understand that various combinations of one or more or a plurality of patch DNA and one or more or a plurality of probe DNA may be used in the methods described herein. The combinations described above are exemplary only and are not intended to be limiting.

According to one aspect, the nucleic acid and the patch may be combined within a reaction volume, such as a liquid reaction volume, and placed under hybridization conditions. Once hybridization of the patch to the nucleic acid has occurred, the patched nucleic acid is then combined with a probe and placed under hybridization conditions to create a patched-probed nucleic acid.

The disclosure contemplates several environments for combining the patched nucleic acid with the probe. According to one aspect, the patched nucleic acid may be combined with the probe in a liquid phase reaction volume where the probe is free within the reaction volume. Once the patched-probed nucleic acid is created, the patched-probed nucleic acid is isolated using methods known to those of skill in the art and analyzed. Such methods include the probe having one member of a binding pair such as avidin-strepavidin bound thereto. The other member of the binding pair may be attached to a substrate. The patched-probed nucleic acid may then be contacted to the substrate, the binding pairs bind and the patched-probed nucleic acid becomes bound to the substrate. The patched-probed nucleic acid may then be isolated from the substrate and analyzed. In addition to avidin-streptavidin, binding pairs include ligand-ligand binding pairs, antibody-antigen binding pairs and the like as known to those of skill in the art. The disclosure of certain binding pairs is exemplary only and is not intended to be limiting.

According to one aspect, the patched nucleic acid may be combined with the probe using microfluidics. A microfluidics system may be used to control and manipulate fluids in a manner to combine the nucleic acid with the patch and the patched nucleic with the probe. Micropumps supply fluids in a continuous manner or are used for dosing. Microvalves determine the flow direction or the mode of movement of pumped liquids. According to one aspect, the microfluidics systems is miniaturized on a single chip, which enhances efficiency and mobility, and reduces sample and reagent volumes. Microfluidics systems include open microfluidics, continuous-flow microfluidics, droplet-based microfluidics, digital microfluidics, or paper-based microfluidics as is known in the art. The nucleic acid, the patch and the probe may be in separate source locations or containers. Each may be fluidly connected to a reaction location or container. A combination of micropumps and/or microvalves can be used to move the nucleic acid and the patch to the reaction location or container and be placed under hybridization conditions. Once the patched nucleic acid is formed, the probe can be introduced by a micropump and/or microvalve combination into the reaction location or container and placed under hybridization conditions to form the patched-probed nucleic acid. The microfluidics system can be designed in a multiplex manner to create a multiplex number of patched-probed nucleic acids. One of skill will readily be able to design certain microfluidics systems as desired to carry out the hybridization reactions described herein. Once the patched-probed nucleic acid is created, it may be transported by a micropump and/or microvalve combination to an analysis location within the microfluidic system or it may be collected from the microfluidic system for further analysis as described herein.

According to one aspect, the patched nucleic acid may be combined with the probe using a microarray to which the probe is bound or other substrate as known in the art to which the probe is bound. Microarrays are known to those of skill in the art and may be commercially manufactured according to desired specifications. According to one aspect, the microarray is an ordered array including a given individual and unique probe at each location of the array. In this manner, a volume of fluid with the patched nucleic acid may be contacted to the entire surface of the array or to portions of the surface of the array under hybridization conditions so that the probe at a given location on the array can hybridize to the patched nucleic acid. In this manner, the hybridization of a plurality of different individual probes may be multiplexed against a given patched nucleic acid. This process may be repeated for each given patch or plurality of patches as desired.

The present disclosure contemplates that use of a microarray is exemplary only and that the disclosure also contemplates that the probe or probes may be attached to other substrates such as columns, beads, tubes, slides, gels and the like as is known to those of skill in the art. The exemplary attachment of DNA probes to substrates, such as silica-based substrates and glass, is known to those of skill in the art and uses readily available and known chemical methods and may be commercially manufactured according to desired specifications.

It is to be understood that the methods described herein need not be utilized with a single patch DNA oligonucleotide and a single probe DNA oligonucleotide. Instead, the disclosure contemplates use of a plurality of patch DNA oligonucleotides that are combined with the nucleic acid and the combination is under hybridization conditions to bind the plurality of patch DNA oligonucleotides to respective binding sites on the nucleic acid. According to one exemplary aspect, a patched or a plurality-patched nucleic acid is contacted to a microarray having a plurality of probe DNA oligonucleotides attached thereto and the microarray and the patched nucleic acid under hybridization conditions to bind the patched nucleic acid to the probe at each probe location of the microarray. According to this aspect, a binding yield is determined for each probe DNA oligonucleotide and compared to a binding yield for each probe DNA oligonucleotide to the naked nucleic acid to identify coupling between patch binding sites and probe binding sites. Coupling between patch binding sites and probe binding sites indicates intramolecular base pairings between the patch binding sites and the probe binding sites. The intramolecular base pairings are analyzed to generate secondary structure of the nucleic acid.

### I. Nucleic Acid

According to one aspect, the term “nucleic acid” generally refers to a nucleic acid polymer. Nucleic acids of the present disclosure are generally understood to be a polymer of nucleotides or ribonucleotides. Nucleic acids may be a polymer of one or more or all of naturally occurring nucleotides or ribonucleotides, synthetic nucleotides or ribonucleotides, peptide nucleotides, locked nucleotides and the like. Naturally occurring nucleotides include the bases adenine, cytosine, guanine, thymine and uracil. A naturally occurring nucleic acid according to the present disclosure for determination of secondary structure includes deoxyribonucleic acid (“DNA”) and ribonucleic acid (“RNA”). Nucleic acids within the scope of the present disclosure may also include one or more or all of a synthetic nucleotide, a peptide nucleotide or a locked nucleotide. Accordingly, the disclosure contemplates determining secondary structure of nucleic acid analogues, peptide nucleic acids, locked nucleic acids, glycol nucleic acids, threose nucleic acids and the like as are known to those of skill in the art. However, it is to be understood that a nucleic acid analogue may have any one or more of and phosphate backbone, pentose sugar or nucleobase altered to create a nucleic acid analogue.

With respect to DNA or RNA for example, primary structure is understood to mean the linear sequence of nucleotides that are linked together by phosphodiester bond. The linear sequence of nucleotides determine the primary structure of DNA or RNA.

With respect to DNA for example, secondary structure is understood to mean the set of interactions between bases, i.e., which parts of strands are bound to each other. The bases in the DNA are classified as purines and pyrimidines. The purines are adenine and guanine. Purines consist of a double ring structure, a six-membered and a five-membered ring containing nitrogen. The pyrimidines are cytosine and thymine. It has a single ring structure, a six-membered ring containing nitrogen. A purine base always pairs with a pyrimidine base (guanine (G) pairs with cytosine (C) and adenine (A) pairs with thymine (T) or uracil (U)). DNA's secondary structure is predominantly determined by base-pairing. In double-stranded DNA, the most common form of the DNA found in cells, two strands of DNA are held together by hydrogen bonds. The nucleotides on one strand base pair with the nucleotides on the other strand. Although the two strands are aligned by hydrogen bonds in base pairs, the stronger forces holding the two strands together are stacking interactions between the bases. These stacking interactions are stabilized by Van der Waals forces and hydrophobic interactions, and show a large amount of local structural variability. There are also two grooves in the double helix, which are called major groove and minor groove based on their relative size. In single-stranded DNA, which is often used in DNA nanotechnology applications, base pairing occurs between complementary regions of a single polynucleotide, as described in more detail for RNA below.

With respect to RNA for example, secondary structure consists of a single polynucleotide. Base pairing in RNA occurs when RNA folds between complementarity regions. Both single- and double-stranded regions are often found in RNA molecules. The four basic elements in the secondary structure of RNA are (1) helices, (2) bulges, (3) loops, and (4) junctions. The antiparallel strands form a helical shape. Bulges and internal loops are formed by separation of the double helical tract on either one strand (bulge) or on both strands (internal loops) by unpaired nucleotides. Stem-loop or hairpin loop is the most common element of RNA secondary structure. Stem-loop is formed when the RNA chains fold back on themselves to form a double helical tract called the ‘stem’, the unpaired nucleotides forms single stranded region called the ‘loop’. A tetraloop is a four-base pairs hairpin RNA structure. There are three common families of tetraloop in ribosomal RNA: UNCG, GNRA, and CUUG (N is one of the four nucleotides and R is a purine). UNCG is the most stable tetraloop. Exemplary RNA for determination of secondary structure include genomes of all single-stranded RNA viruses, messenger RNA (mRNA), long non-coding RNA (lncRNA), ribosomal RNA (rRNA) and other ribozymes, and certain transposable elements, such as long terminal repeat (LTR) and long interspersed nuclear element (LINE) retrotransposons.

According to one aspect, the nucleic acid may be any nucleic acid where it is desirable to resolve the secondary structure. The nucleic acid may any length. Suitable lengths include from 10 nucleotides to 50,000 nucleotides.

According to one aspect, exemplary lengths for a DNA nucleic acid include those known to one of skill based on the primary structure of known DNA molecules. These lengths can be easily determined in the literature. Long DNA refers to any DNA being 200 nucleotides or greater in length. Examples of long DNA include the genomes of all single-stranded DNA viruses and certain DNA strands used for DNA nanotechnology, such as the template strands used in DNA origami.

According to one aspect, exemplary lengths for an RNA nucleic acid include those know to one of skill based on the primary structure of known RNA molecules. These lengths can be easily determined in the literature. According to one aspect, a long nucleotide, such as a long RNA has 200 nucleotides or greater. According to one aspect, the RNA is between 200 nucleotides and 50,000 nucleotides in length, is between 200 nucleotides and 30,000 nucleotides in length, is between 200 nucleotides and 20,000 nucleotides in length, is between 200 nucleotides and 10,000 nucleotides in length, is between 500 nucleotides and 5,000 nucleotides in length, is between 1000 nucleotides and 3,000 nucleotides in length, is between 200 nucleotides and 5,000 nucleotides in length, is between 200 nucleotides and 3,000 nucleotides in length, is between 500 nucleotides and 10,000 nucleotides in length, or is between 500 nucleotides and 3,000 nucleotides in length. Examples of long RNA include the genomes of all single-stranded RNA viruses, messenger RNA (mRNA), long non-coding RNA (lncRNA), ribosomal RNA (rRNA) and other ribozymes, and certain transposable elements, such as long terminal repeat (LTR) and long interspersed nuclear element (LINE) retrotransposons.

According to one aspect, the nucleic acid may be present in any suitable amount. However, the methods described herein are especially suited to low amounts of nucleic acid, such as the nucleic acid (which may be RNA) being present in an amount of 1 μg or less.

### II. Patch and Probe Oligonucleotides

According to certain aspects, patch oligonucleotides are designed such that their sequences are complementary to regions of the nucleic acid molecule. In the example experiment, a nucleic acid, such as a long RNA sequence is divided into contiguous tiles, each of which is perfectly complementary to a separate patch oligonucleotide. The probe oligonucleotides are also designed such that their sequences are complementary to regions of the lone RNA sequence. According to one aspect, each patch designed to tile a given RNA may be the same length. According to one aspect, each patch designed to tile a given RNA may be different lengths. According to one aspect, each probe designed to tile a given RNA may be the same length. According to one aspect, each probe designed to tile a given RNA may be different lengths. According to one aspect, each probe oligonucleotide is the same length and together they are complementary to every possible contiguous region of the RNA of that length.

According to one aspect, multiple patch and probe oligonucleotides are designed and used systematically as described herein to reveal secondary structure of the nucleic acid. The patch oligonucleotide and probe oligonucleotide can be used in an amount sufficient to bind to their respective binding sites which can be determined by those of skill in the art.

Exemplary lengths of patch and probe oligonucleotides are those that are sufficiently shorter than the nucleic acid so as to serve as patch and probe oligonucleotides. Exemplary ranges include at least 2 nucleotides in length, between 2 nucleotides and 200 nucleotides in length, between 2 nucleotides and 50 nucleotides, between 5 and 50 nucleotides, between 5 and 25 nucleotides, between 10 and 30 nucleotides, between 10 and 50 nucleotides, between 15 nucleotides and 25 nucleotides in length, between 12 and 48 nucleotides or between 12 and 24 nucleotides.

The patch and probe oligonucleotides may be RNA or DNA or include one or more or all of a synthetic nucleotide, a peptide nucleotide or a locked nucleotide, or other synthetic analogs known to those with skill in the art.

### III. Folding Algorithms

The disclosure contemplates the use of folding algorithms to supplement methods of determining secondary structure. However, it is to be understood that folding algorithms are not required for use with the disclosed methods as the disclosed methods are used to analyze or determine secondary structure directly and without the use of a folding algorithm. However, should one of skill choose to use a folding algorithm in conjunction with the disclosed methods, exemplary folding algorithms known to those of skill in the art include MFOLD, ViennaRNA, NUPACK, and RNAstructure. In general and with respect to determining secondary structure of RNA, folding algorithms are used to enumerate all possible combinations of base pairs within a linear RNA sequence. Compatible combinations of base pairs are grouped into a set of structures, and the likelihood of structures within this set are ordered by their estimated free energies. The structure free energies are calculated by adding up individual contributions from each motif within the structure, using energy parameters that were previously empirically measured. Although folding algorithms have been used to predict structures of short (<200 nucleotides) RNAs, folding algorithms may not be useful with longer molecules, in part due to the additive error in the free energy calculations. The structures with the lowest free energies are predicted to be the most likely to form, but the calculated free energies of long RNA molecules can significantly differ from their true energies. However, RNA folding algorithms can be used in conjunction with the disclosed methods in principle to either increase the accuracy of structure prediction or increase the resolution of the algorithm-free image. When using a folding algorithm, the described methods which are sensitive to the presence of multiple structures can help eliminate structures within the set of calculated structures that are not detectable, thereby constraining the set of possible structures. Vice versa, the set of possible folded structures predicted by folding algorithms can be compared directly with data generated by the disclosed methods.

### EXAMPLES

The following examples are set forth as representative of the present invention. These examples are not to be construed as limiting the scope of the invention as these and other equivalent embodiments will be apparent in view of the present disclosure and appended claims.

### Example I

The following describes a method of the present disclosure using the 1,058-base-long RNA genome of satellite tobacco mosaic virus (STMV), whose secondary structure has been determined using a variety of biophysical techniques (Athavale et al., Plos One 8: e54384).

AlexaFluor-546-labeled RNA samples were transcribed from linearized DNA plasmid templates using a TranscriptAid T7 High Yield Transcription Kit (Thermo Scientific). A small amount of AF-546 UTP was incorporated into the transcription mixture. Transcripts were purified using acid phenol chloroform, washed in a centrifuge filter, redissolved into pure water, and stored at −80° C. Product and label yields were measured by UV-Vis on a Nanodrop spectrophotometer.

Hybridization conditions to hybridize patch DNA oligonucleotides to an RNA nucleic acid are as follows. 1 μg of RNA was diluted to 10 nM in (50 mM Tris, 1 mM EDTA, 1 M NaCl, pH 7.0, 0.5% Tween-20) and combined with a 1:1 concentration ratio of patch DNA oligomers (24-base long). The RNA-DNA mixtures were heated to 90° C. and then cooled to 4° C. at a rate of −1° C./s.

Hybridization conditions to hybridize probe DNA oligonucleotides attached to a microarray to a patched RNA nucleic acid are as follows. The patched RNA was added to the microarray slide containing the probe DNA oligomers for 100 minutes at 37° C.

Each microarray contained 48 individual subarrays. The microarrays were subsequently washed for 1 minute with the same buffer used in the hybridization and dried completely. Images of the arrays were taken using an Agilent SureScan Microarray Scanner. The scanned microarray images were processed using MATLAB. The boundaries of individual subarrays were picked manually, and a rectangular grid was placed on the image that defines the spot boundaries. Spot centroids were found, and the spot intensities were calculated by integrating the pixels within the spot.

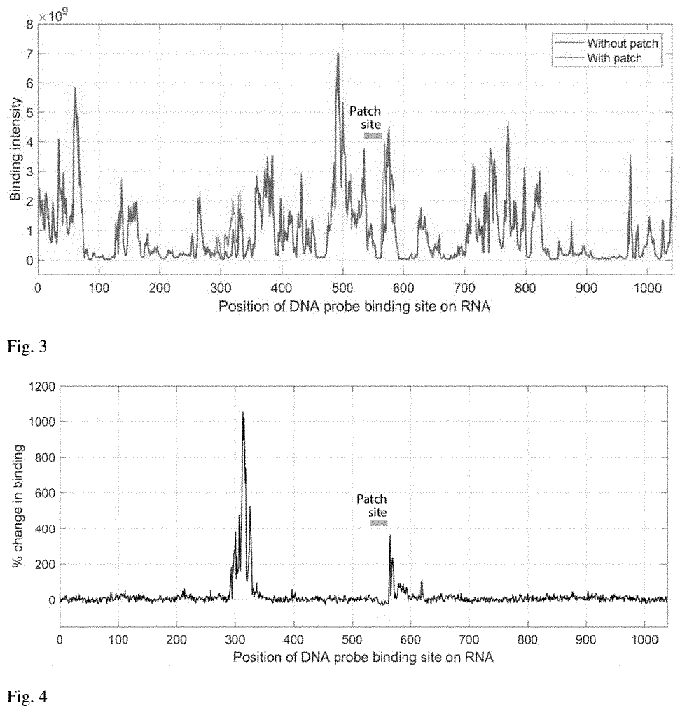

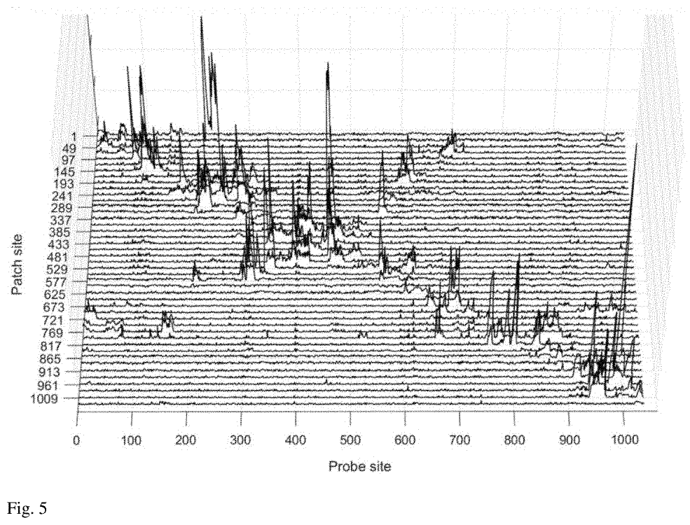

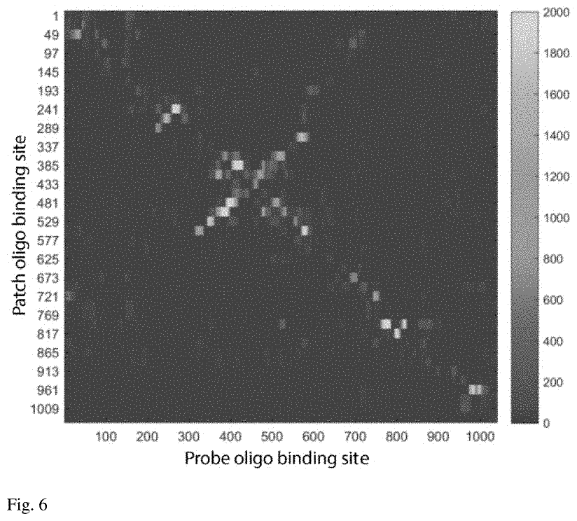

FIG. 2 illustrates that 44 complementary 24-base-long patch DNA strands were designed that collectively tiled the STMV RNA primary sequence from base 1 to base 1,056. In parallel, the STMV RNA was hybridized to each of the 44 patch DNA strands. DNA microarrays containing all approximately 1,058 possible complementary 12- and 24-base-long probe DNA strands were used to measure the yield of probe binding. As shown in FIG. 3, a binding spectrum was created by plotting binding yield for each probe oligo arranged in ascending order based on the position of the probe binding site along the RNA primary sequence. As shown in FIG. 4, the change induced by each patch DNA strand was directly measured by plotting the difference between the binding spectrum obtained with a given patch DNA strand and the binding spectrum obtained without patch DNA. The pattern produced by aligning each difference plot according to the position of the patch oligo binding site along the RNA primary sequence (see FIG. 5) can be viewed as a 2D heat map (see FIG. 6). This pattern, as described above, reflects couplings between patch and probe binding sites on the RNA, thereby determining intramolecular RNA base pairing between these binding sites.

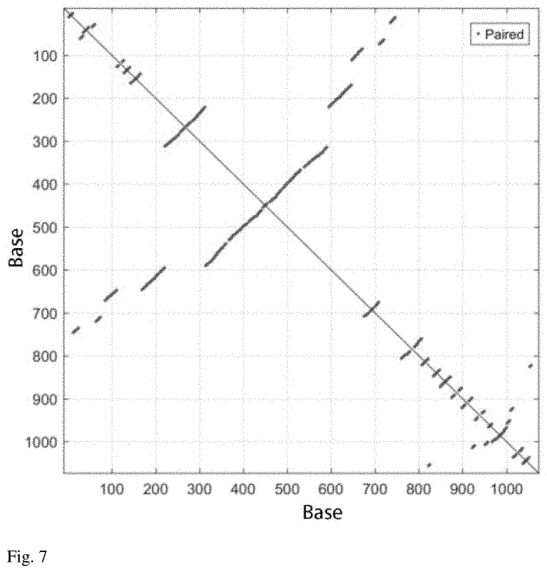

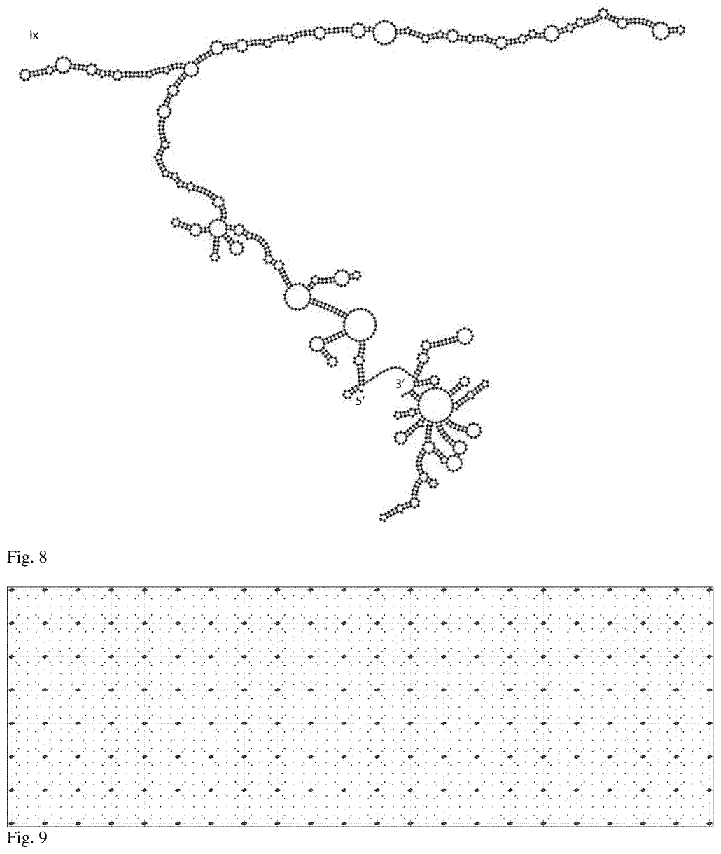

Comparison of the 2D heat map (see FIG. 6) with the adjacency matrix (see FIG. 7) for the consensus secondary structure for STMV RNA (ix) generates a readily interpretable image of the RNA secondary structure directly from sequence data alone, without the need of RNA folding algorithms.

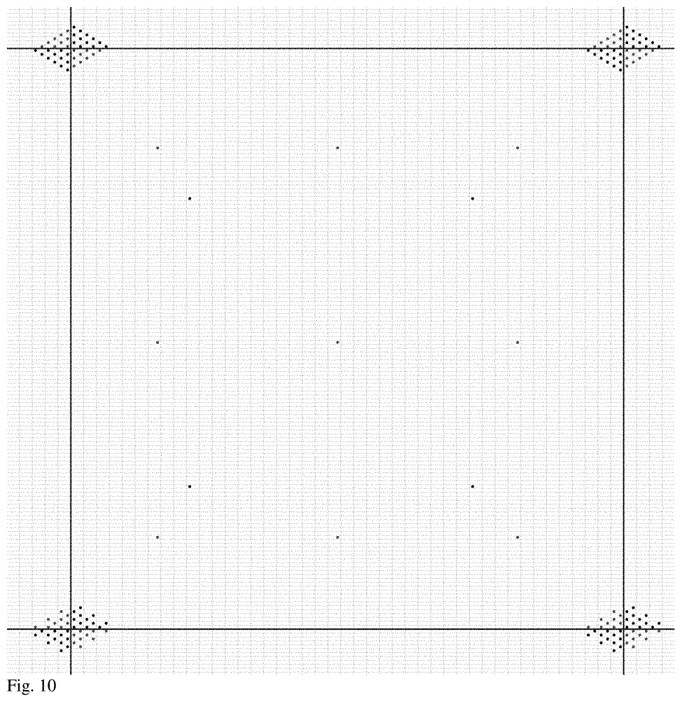

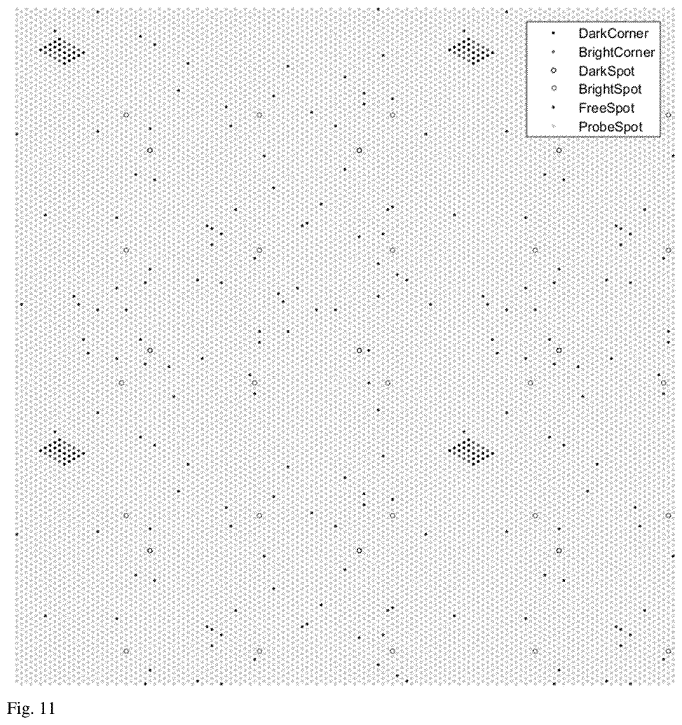

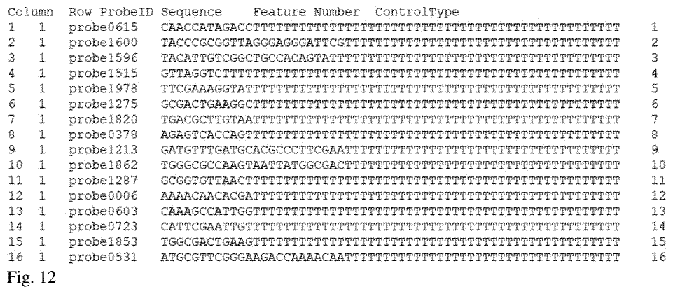

DNA microarrays are manufactured by Agilent Technologies (Supplier Item G4860A, SurePrint G3 Custom GE 1×1M Microarray). Within each 1 million-spot array (see FIG. 9), small rectangular subarrays can be designed that contain triplicate sequences of all 12- and 24-base long probe DNA oligomers (see FIGS. 10 and 11). Probe sequences are the reverse complement to every site on the RNA, and the spatial coordinates of each spot are distributed randomly within a subarray (see FIGS. 10, 11, and 12). Patterns of dark and light spots of the subarrays are designed as visual aids in the image analysis process. Some of these features are located in the corners and some are distributed uniformly throughout the subarray. In the example experiment on 1058-base long STMV RNA, 147 full subarrays fit into the 1M array, where each subarray had (149 rows×43 columns).

Those having skill in the art, with the knowledge gained from the present disclosure, will recognize that various changes can be made to the disclosed methods in attaining these and other advantages, without departing from the scope of the present invention. Accordingly, it should be understood that the features described herein are susceptible to changes or substitutions. The specific embodiments illustrated and described herein are for illustrative purposes only, and not limiting of the invention as set forth in the appended claims.

### Example II

### Embodiments

Aspects of the present disclosure are directed to a method of analyzing secondary structure of a nucleic acid. The method includes combining the nucleic acid and a patch oligonucleotide and placing under hybridization conditions to hybridize the patch oligonucleotide to a patch binding site to form a patched nucleic acid, combining the patched nucleic acid with a probe oligonucleotide and placing under hybridization conditions to hybridize the probe oligonucleotide to a probe binding site to form a patched-probed nucleic acid, determining a first binding yield of the probe oligonucleotide to the patched nucleic acid, comparing the first binding yield to a second binding yield of the probe oligonucleotide to a naked nucleic acid, whereby the first binding yield being greater than or differing significantly from the second binding yield indicates that the patch binding site and the probe binding site are coupled and located on a common secondary structural element of the nucleic acid; and whereby the first binding yield and the second binding yield being similar indicates that the first binding site and the second binding site are independent and located on different secondary structural elements of the nucleic acid. According to one aspect, the nucleic acid is DNA or RNA. According to one aspect, the nucleic acid is a genome of a single-stranded RNA virus, a messenger RNA (mRNA), a long non-coding RNA (lncRNA), a ribosomal RNA (rRNA), a ribozyme, a transposable elements, a long terminal repeat (LTR) or a long interspersed nuclear element (LINE) retrotransposon. According to one aspect, binding to the patch binding site by the patch DNA oligonucleotide enhances binding of the probe oligonucleotide to the probe binding site. According to one aspect, the patch binding site and the probe binding site comprise complementary base pairings. According to one aspect, the nucleic acid is long RNA having 200 nucleotides in length or greater, between 200 nucleotides and 50,000 nucleotides in length, between 200 nucleotides and 30,000 nucleotides in length, between 200 nucleotides and 20,000 nucleotides in length, between 200 nucleotides and 10,000 nucleotides in length, between 500 nucleotides and 5,000 nucleotides in length, between 1000 nucleotides and 3,000 nucleotides in length, between 200 nucleotides and 5,000 nucleotides in length, between 200 nucleotides and 3,000 nucleotides in length, between 500 nucleotides and 10,000 nucleotides in length, or between 500 nucleotides and 3,000 nucleotides in length. According to one aspect, the first patch oligonucleotide and the second probe oligonucleotide are each at least 2 nucleotides in length, between 2 nucleotides and 200 nucleotides in length, between 2 nucleotides and 200 nucleotides in length, between 5 and 50 nucleotides in length, between 5 and 25 nucleotides in length, between 10 and 30 nucleotides in length, between 10 and 50 nucleotides in length, between 15 nucleotides and 25 nucleotides in length, between 12 and 48 nucleotides in length or between 12 and 24 nucleotides in length. According to one aspect, the nucleic acid and the patch oligonucleotide are combined in a reaction volume and the combination is under hybridization conditions to bind the patch oligonucleotide to a patch binding site of the nucleic acid. According to one aspect, the probe oligonucleotide is attached to a microarray and the patched nucleic acid is contacted to the microarray and the microarray is under hybridization conditions. According to one aspect, a plurality of probe oligonucleotides are attached to a microarray and the patched nucleic acid is contacted to the microarray and the microarray is under hybridization conditions. According to one aspect, a plurality of patch oligonucleotides are combined with the nucleic acid and the combination is under hybridization conditions to bind the plurality of patch oligonucleotides to respective patch binding sites of the nucleic acid to form a plurality-patched nucleic acid. According to one aspect, a plurality of patch oligonucleotides are combined with the nucleic acid and the combination is under hybridization conditions to bind the plurality of patch oligonucleotides to respective patch binding sites of the nucleic acid to form a plurality-patched nucleic acid, thereafter the plurality-patched nucleic acid is contacted to a microarray having a plurality of probe oligonucleotides attached thereto and the microarray is hybridization conditions. According to one aspect, a binding yield is determined for each probe oligonucleotide. According to one aspect, a binding yield is determined for each probe oligonucleotide and compared to a binding yield for each probe oligonucleotide to the nucleic acid without the patch oligonucleotide to identify coupling between patch binding sites and probe binding sites. According to one aspect, the coupling between first binding sites and second binding sites indicates intramolecular base pairings between the patch binding sites and the probe binding sites. According to one aspect, the intramolecular base pairings are analyzed to generate secondary structure of the nucleic acid. According to one aspect, the nucleic acid is RNA. According to one aspect, an RNA folding algorithm is used. According to one aspect, the method is carried out with the proviso that an RNA folding algorithm is not used. According to one aspect, the nucleic acid and the patch oligonucleotide are combined using a microfluidic system. According to one aspect, the patched nucleic acid and the probe oligonucleotide are combined using a microfluidic system. According to one aspect, the patch oligonucleotide is DNA or RNA. According to one aspect, the probe oligonucleotide is DNA or RNA. According to one aspect, a plurality of patch oligonucleotides and a plurality of probe oligonucleotides are used to determine binding yields for a set of combinations of patch oligonucleotides and probe oligonucleotides.

According to one aspect, the disclosure provides a combination comprising a nucleic acid, a patch oligonucleotide and a plurality of probe oligonucleotides. According to one aspect, the plurality of probe oligonucleotides are attached to a microarray.

What is claimed is:

1. A method of analyzing secondary structure of a nucleic acid comprising
combining the nucleic acid and a patch oligonucleotide and placing under hybridization conditions to hybridize the patch oligonucleotide to a patch binding site to form a patched nucleic acid;
combining the patched nucleic acid with a probe oligonucleotide and placing under hybridization conditions to hybridize the probe oligonucleotide to a probe binding site to form a patched-probed nucleic acid;
determining a first binding yield of the probe oligonucleotide to the patched nucleic acid;
comparing the first binding yield to a second binding yield of the probe oligonucleotide to a naked nucleic acid;
whereby the first binding yield being greater than or differing significantly from the second binding yield indicates that the patch binding site and the probe binding site are coupled and located on a common secondary structural element of the nucleic acid; and
whereby the first binding yield and the second binding yield being similar indicates that the first binding site and the second binding site are independent and located on different secondary structural elements of the nucleic acid.

2. The method of claim 1 wherein the nucleic acid is DNA or RNA.

3. The method of claim 1 wherein the nucleic acid is a genome of a single-stranded RNA virus, a messenger RNA (mRNA), a long non-coding RNA (lncRNA), a ribosomal RNA (rRNA), a ribozyme, a transposable elements, a long terminal repeat (LTR) or a long interspersed nuclear element (LINE) retrotransposon.

4. The method of claim 1 wherein binding to the patch binding site by the patch DNA oligonucleotide enhances binding of the probe oligonucleotide to the probe binding site.

5. The method of claim 1 wherein the patch binding site and the probe binding site comprise complementary base pairings.

6. The method of claim 1 wherein the nucleic acid is long RNA having 200 nucleotides in length or greater, between 200 nucleotides and 50,000 nucleotides in length, between 200 nucleotides and 30,000 nucleotides in length, between 200 nucleotides and 20,000 nucleotides in length, between 200 nucleotides and 10,000 nucleotides in length, between 500 nucleotides and 5,000 nucleotides in length, between 1000 nucleotides and 3,000 nucleotides in length, between 200 nucleotides and 5,000 nucleotides in length, between 200 nucleotides and 3,000 nucleotides in length, between 500 nucleotides and 10,000 nucleotides in length, or between 500 nucleotides and 3,000 nucleotides in length.

7. The method of claim 1 wherein the first patch oligonucleotide and the second probe oligonucleotide are each at least 2 nucleotides in length, between 2 nucleotides and 200 nucleotides in length, between 2 nucleotides and 200 nucleotides in length, between 5 and 50 nucleotides in length, between 5 and 25 nucleotides in length, between 10 and 30 nucleotides in length, between 10 and 50 nucleotides in length, between 15 nucleotides and 25 nucleotides in length, between 12 and 48 nucleotides in length or between 12 and 24 nucleotides in length.

8. The method of claim 1 wherein the nucleic acid and the patch oligonucleotide are combined in a reaction volume and the combination is under hybridization conditions to bind the patch oligonucleotide to a patch binding site of the nucleic acid.

9. The method of claim 1 wherein the probe oligonucleotide is attached to a microarray and the patched nucleic acid is contacted to the microarray and the microarray is under hybridization conditions.

10. The method of claim 1 wherein a plurality of probe oligonucleotides are attached to a microarray and the patched nucleic acid is contacted to the microarray and the microarray is under hybridization conditions.

11. The method of claim 1 wherein a plurality of patch oligonucleotides are combined with the nucleic acid and the combination is under hybridization conditions to bind the plurality of patch oligonucleotides to respective patch binding sites of the nucleic acid to form a plurality-patched nucleic acid.

12. The method of claim 1 wherein a plurality of patch oligonucleotides are combined with the nucleic acid and the combination is under hybridization conditions to bind the plurality of patch oligonucleotides to respective patch binding sites of the nucleic acid to form a plurality-patched nucleic acid;
thereafter the plurality-patched nucleic acid is contacted to a microarray having a plurality of probe oligonucleotides attached thereto and the microarray is hybridization conditions.

13. The method of claim 11 wherein a binding yield is determined for each probe oligonucleotide.

14. The method of claim 11 wherein a binding yield is determined for each probe oligonucleotide and compared to a binding yield for each probe oligonucleotide to the nucleic acid without the patch oligonucleotide to identify coupling between patch binding sites and probe binding sites.

16. The method of claim 14 wherein the coupling between first binding sites and second binding sites indicates intramolecular base pairings between the patch binding sites and the probe binding sites.

16. The method of claim 15 wherein the intramolecular base pairings are analyzed to generate secondary structure of the nucleic acid.

17. The method of claim 1 wherein the nucleic acid is RNA.

18. The method of claim 1 further comprising use of an RNA folding algorithm.

19. The method of claim 1 with the proviso that an RNA folding algorithm is not used.

20. The method of claim 1 where the nucleic acid and the patch oligonucleotide are combined using a microfluidic system.

21. The method of claim 1 wherein the patched nucleic acid and the probe oligonucleotide are combined using a microfluidic system.

22. The method of claim 1 wherein the patch oligonucleotide is DNA or RNA.

23. The method of claim 1 wherein the probe oligonucleotide is DNA or RNA.

24. The method of claim 1 wherein a plurality of patch oligonucleotides and a plurality of probe oligonucleotides are used to determine binding yields for a set of combinations of patch oligonucleotides and probe oligonucleotides.

25. A combination comprising a nucleic acid, a patch oligonucleotide and a plurality of probe oligonucleotides.

26. The combination of claim 25 wherein the plurality of probe oligonucleotides are attached to a microarray.


SEQUENCE LISTING (16 sequences)

SEQ ID NO: 1 - DNA, 60 bp, Artificial
  probe0615
caaccatagacctttttttttttttttttttttttttttttttttttttttttttttttt

SEQ ID NO: 2 - DNA, 60 bp, Artificial
  probe1600
tacccgcggttagggagggattcgtttttttttttttttttttttttttttttttttttt

SEQ ID NO: 3 - DNA, 60 bp, Artificial
  probe1596
tacattgtcggctgccacagtatttttttttttttttttttttttttttttttttttttt

SEQ ID NO: 4 - DNA, 60 bp, Artificial
  probe1515
gttaggtctttttttttttttttttttttttttttttttttttttttttttttttttttt

SEQ ID NO: 5 - DNA, 60 bp, Artificial
  probe1978
ttcgaaaggtattttttttttttttttttttttttttttttttttttttttttttttttt

SEQ ID NO: 6 - DNA, 60 bp, Artificial
  probe1275
gcgactgaaggctttttttttttttttttttttttttttttttttttttttttttttttt

SEQ ID NO: 7 - DNA, 60 bp, Artificial
  probe1820
tgacgcttgtaatttttttttttttttttttttttttttttttttttttttttttttttt

SEQ ID NO: 8 - DNA, 60 bp, Artificial
  probe0378
agagtcaccagttttttttttttttttttttttttttttttttttttttttttttttttt

SEQ ID NO: 9 - DNA, 60 bp, Artificial
  probe1213
gatgtttgatgcacgcccttcgaatttttttttttttttttttttttttttttttttttt

SEQ ID NO: 10 - DNA, 60 bp, Artificial
  probe1862
tgggcgccaagtaattatggcgactttttttttttttttttttttttttttttttttttt

SEQ ID NO: 11 - DNA, 60 bp, Artificial
  probe1287
gcggtgttaacttttttttttttttttttttttttttttttttttttttttttttttttt

SEQ ID NO: 12 - DNA, 60 bp, Artificial
  probe0006
aaaacaacacgatttttttttttttttttttttttttttttttttttttttttttttttt

SEQ ID NO: 13 - DNA, 60 bp, Artificial
  probe0603
caaagccattggtttttttttttttttttttttttttttttttttttttttttttttttt

SEQ ID NO: 14 - DNA, 60 bp, Artificial
  probe0723
cattcgaattgttttttttttttttttttttttttttttttttttttttttttttttttt

SEQ ID NO: 15 - DNA, 60 bp, Artificial
  probe1853
tggcgactgaagtttttttttttttttttttttttttttttttttttttttttttttttt

SEQ ID NO: 16 - DNA, 60 bp, Artificial
  probe0531
atgcgttcgggaagaccaaaacaatttttttttttttttttttttttttttttttttttt